(19) United States
(12) Patent Application Publication
(10) Pub. No.: US 2011/0201656 A1
(43) Pub. Date: Aug. 18, 2011

(54) NOVEL DIPHENYL 1,2,3-TRIAZOLE DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Kobenhavn N (DK); Dan Peters, Malmo (SE)

(73) Assignee: NEUROSEARCH A/S, Ballerup (DK)

(21) Appl. No.: 13/057,957

(22) PCT Filed: Aug. 3, 2009
(86) PCT No.: PCT/EP09/59992

§ 371 Date: May 5, 2011

Related U.S. Application Data
(60) Provisional application No. 61/087,428, filed on Aug. 8, 2008.

(30) Foreign Application Priority Data
Aug. 8, 2008 (DK) PA 2008 01082

(51) Int. Cl. A61K 31/4192; C07D 249/06; A61P 25/00; A61P 25/22; A61P 25/28; A61P 25/16; A61P 25/24; A61P 25/18; A61P 3/04; A61P 25/20; A61P 25/02; A61P 25/08; A61P 9/12; A61P 9/06; A61P 21/00; A61P 1/12; A61P 11/06; A61P 15/00; A61P 5/00; A61P 25/04; A61P 25/06; A61P 29/00; A61P 17/00; A61P 17/10; A61P 25/36; A61P 25/30

(52) U.S. Cl. 514/359; 548/255

(57) ABSTRACT

This invention relates to novel diphenyl 1,2,3-triazole derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

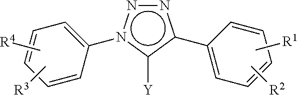

### TECHNICAL FIELD

This invention relates to novel diphenyl 1,2,3-triazole derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

US 2009 069569 describes a method of producing 1- and/or 4-substituted 1,2,3-triazole compounds. However, any biological activity is not reported.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labeled or unlabelled form.

In its first aspect the invention provides diphenyl 1,2,3-triazole derivatives of Formula I

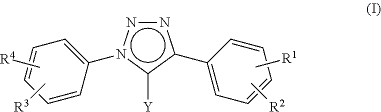

a stereoisomer thereof or a mixture of its stereoisomers, or a pharmaceutically acceptable salt thereof, wherein

Y represents hydrogen, halo, alkyl, halo-alkyl, hydroxy-alkyl or aminoalkyl; and

R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

In a second aspect the invention provides pharmaceutical compositions comprising a therapeutically effective amount of the diphenyl 1,2,3-triazole derivative of the invention, or a pharmaceutically acceptable addition salt thereof, together with at least one pharmaceutically acceptable carrier or diluent.

Viewed from another aspect the invention relates to the use of the diphenyl 1,2,3-triazole derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the diphenyl 1,2,3-triazole derivative of the invention.

Other objects of the invention will be apparent to the person skilled in the art from the following detailed description and examples.

### DETAILED DISCLOSURE OF THE INVENTION

### Diphenyl 1,2,3-Triazole Derivatives

In its first aspect the invention provides diphenyl 1,2,3-triazole derivatives of Formula I

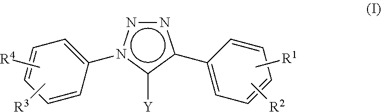

a stereoisomer thereof or a mixture of its stereoisomers, or a pharmaceutically acceptable salt thereof, wherein

Y represents hydrogen, halo, alkyl, halo-alkyl, hydroxy-alkyl or amino-alkyl; and

R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

In a more preferred embodiment, the diphenyl 1,2,3-triazole derivative of the invention is a compound of Formula Ian

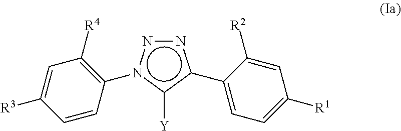

a stereoisomer thereof or a mixture of its stereoisomers, or a pharmaceutically acceptable salt thereof, wherein Y, R¹, R², R³ and R⁴, are as defined in above.

In another preferred embodiment the invention provides diphenyl 1,2,3-triazole derivatives of Formula I or Ia, a stereoisomer thereof or a mixture of its stereoisomers, or a pharmaceutically acceptable salt thereof, wherein

Y represents alkyl, halo-alkyl, hydroxy-alkyl or amino-alkyl; and

R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

In a preferred embodiment the diphenyl 1,2,3-triazole derivative of the invention is a compound of Formula I, wherein Y represents alkyl, halo-alkyl, hydroxy-alkyl or amino-alkyl.

In a more preferred embodiment Y represents alkyl, and in particular methyl.

In another more preferred embodiment Y represents halo-alkyl, and in particular halomethyl, most preferred bromomethyl.

In a third more preferred embodiment Y represents hydroxy-alkyl, and in particular hydroxymethyl.

In a fourth more preferred embodiment Y represents amino-alkyl, and in particular aminomethyl.

In a fifth more preferred embodiment Y represents hydrogen.

In a sixth more preferred embodiment Y represents halo, and in particular chloro.

In another preferred embodiment the diphenyl 1,2,3-triazole derivative of the invention is a compound of Formula I, wherein R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

In a more preferred embodiment R¹, R², R³ and R⁴, independently of each other, represent a substituent selected from the group consisting of hydrogen, halo, and in particular fluoro or chloro, trifluoromethyl, alkoxy, and in particular methoxy, and hydroxy.

In another more preferred embodiment one of R¹ and R² represents hydrogen; and the other of R¹ and R² represents hydroxy or alkoxy, and in particular methoxy.

In a third more preferred embodiment R¹ represents hydroxy or alkoxy, and in particular methoxy; and R² represents hydrogen.

In a fourth more preferred embodiment R¹ represents hydroxy; and R² represents hydrogen.

In a fifth more preferred embodiment R¹ represents alkoxy, and in particular methoxy; and R² represents hydrogen.

In a sixth more preferred embodiment one of R³ and R⁴ represents halo, and in particular fluoro or chloro; and the other of R³ and R⁴ represents trifluoromethyl.

In a seventh more preferred embodiment R³ represents trifluoromethyl; and R⁴ represents halo, and in particular fluoro or chloro.

In an eight more preferred embodiment one of R¹ and R² represents hydroxy or alkoxy, and in particular methoxy; and the other of R¹ and R² represents halo, and in particular chloro.

In a ninth more preferred embodiment R¹ represents hydroxy or alkoxy, and in particular methoxy; and R² represents halo, and in particular chloro.

In a most preferred embodiment the diphenyl 1,2,3-triazole derivative of the invention is

1-(2-Fluoro-4-trifluoromethyl-phenyl)-4-(4-methoxy-phenyl)-5-methyl-1H-[1,2,3]triazole;

1-(2-Chloro-4-trifluoromethyl-phenyl)-4-(4-methoxy-phenyl)-5-methyl-1H-[1,2,3]triazole;

4-[1-(2-Chloro-4-trifluoromethyl-phenyl)-5-methyl-1H-[1,2,3]triazol-4-yl]-phenol;

5-Bromomethyl-1-(2-chloro-4-trifluoromethyl-phenyl)-4-(4-methoxy-phenyl)-1H-[1,2,3]triazole;

C-[3-(2-Chloro-4-trifluoromethyl-phenyl)-5-(4-methoxy-phenyl)-3H-[1,2,3]triazol-4-yl]-methylamine;

4-[5-Aminomethyl-1-(2-chloro-4-trifluoromethyl-phenyl)-1H-[1,2,3]triazol-4-yl]-phenol;

[3-(2-Chloro-4-trifluoromethyl-phenyl)-5-(4-methoxy-phenyl)-3H-[1,2,3]triazol-4-yl]-methanol;

4-[1-(2-Chloro-4-trifluoromethyl-phenyl)-5-hydroxymethyl-1H-[1,2,3]triazol-4-yl]-phenol;

1-(2-Fluoro-4-trifluoromethyl-phenyl)-4-(4-methoxy-phenyl)-1H-[1,2,3]triazole;

4-[1-(2-Fluoro-4-trifluoromethyl-phenyl)-1H-[1,2,3]triazol-4-yl]-phenol;

5-Chloro-4-(2-chloro-4-methoxy-phenyl)-1-(2-fluoro-4-trifluoromethyl-phenyl)-1H-[1,2,3]triazole; or

3-Chloro-4-[5-chloro-1-(2-fluoro-4-trifluoromethyl-phenyl)-1H-[1,2,3]triazol-4-yl]-phenol;

a stereoisomer thereof or a mixture of its stereoisomers, or a pharmaceutically acceptable salt thereof.

Any combination of two or more of the embodiments described herein is considered within the scope of the present invention.

### DEFINITION OF SUBSTITUENTS

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O-” group, wherein alkyl is as defined above. Examples of preferred alkoxy groups of the invention include methoxy and ethoxy.

In the context of this invention a hydroxy-alkyl group designates an alkyl group as defined above, which alkyl group is substituted with one or more hydroxy groups. Examples of preferred hydroxy-alkyl groups of the invention include hydroxy-methyl, 2-hydroxy-ethyl, 3-hydroxy-propyl, 4-hydroxy-butyl, 5-hydroxy-pentyl and 6-hydroxy-hexyl.

In the context of this invention a halo-alkyl group designates an alkyl group as defined above, which alkyl group is mono-substituted with halo, and which halo is as defined above. Examples of preferred halo-alkyl groups of the invention include halo-methyl, 2-halo-ethyl, 3-halo-propyl, 4-halo-butyl, 5-halo-pentyl and 6-halo-hexyl.

In the context of this invention an amino-alkyl group designates an alkyl group as defined above, which alkyl group is mono-substituted with amino. Examples of preferred amino-alkyl groups of the invention include amino-methyl, 2-amino-ethyl, 3-amino-propyl, 4-amino-butyl, 5-amino-pentyl and 6-amino-hexyl.

### Pharmaceutically Acceptable Salts

The diphenyl 1,2,3-triazole derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a diphenyl 1,2,3-triazole derivative of the invention include alkali metal salts, such as the sodium salt of a compound of the invention containing a carboxy group.

### Steric Isomers

It will be appreciated by those skilled in the art that the diphenyl 1,2,3-triazole derivatives of the present invention may exist in different stereo isomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Producing Diphenyl 1,2,3-Triazole Derivatives

The diphenyl 1,2,3-triazole derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show a pronounced nicotinic acetylcholine a7 receptor subtype selectivity.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of diphenyl 1,2,3-triazole derivative of the invention.

While a diphenyl 1,2,3-triazole derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the diphenyl 1,2,3-triazole derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The diphenyl 1,2,3-triazole derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a diphenyl 1,2,3-triazole derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

1-(2-Fluoro-4-trifluoromethyl-phenyl)-4-(4-methoxy-phenyl)-5-methyl-1H-[1,2,3]triazole (Compound 1)

To a stirred and ice-cooled solution of sodium methoxide (0.296 g, 5.4848 mmol) in methanol (25 ml), 4-methoxyphenylacetone (0.661 g, 4.0222 mmol) and 1-azido-2-fluoro-4-trifluoromethyl-benzene (0.750 g, 3.6565 mmol) are added portion-wise under a nitrogen atmosphere. The reaction mixture is allowed to attain room temperature spontaneously overnight, concentrated in vacuo, water added and extracted with ethyl acetate (3×80 ml). The combined organic layers are dried over MgSO₄, filtered and evaporated, to give a dark brown solid (1.185 g, 92% mass balance). This crude material is purified by column chromatography over silica gel (230-400 mesh) eluting with 9% ethyl acetate in petroleum ether, to afford the title compound as a white solid (0.600 mg, 47% yield). M.p. 153.8-154.9° C. LC-ESI-HRMS of [M+H]+ shows 352.107 Da. Calc. 352.107299 Da, dev. −0.8 ppm.

1-(2-Chloro-4-trifluoromethyl-phenyl)-4-(4-methoxy-phenyl)-5-methyl-1H-[1,2,3]triazole (Compound 2)

To a stirred and ice-cooled solution of sodium methoxide (2.750 g, 50.9036 mmol) in methanol (200 ml), commercial 4-methoxyphenylacetone (6.100 g, 37.1492 mmol) and 1-azido-2-chloro-4-trifluoromethyl-benzene (7.500 g, 33.8495 mmol) are added under a nitrogen atmosphere. The reaction mixture is kept at 0° C. for 1 h, then allowed to attain spontaneously room temperature, and finally refluxed overnight. The reaction mixture is concentrated, water added and extracted with ethyl acetate (3×500 ml). The combined organic layers are dried over MgSO₄, filtered and evaporated, to afford a dark brown gummy material (˜12.3 g, 98% mass balance). The crude residue is purified by column chromatography over silica gel (230-400 mesh), eluting with 2-9% ethyl acetate in petroleum ether, to obtain the title compound as a yellow solid (4.100 g, 33% yield). LCMS: >99% UV analysis, MH⁺=368.

4-[1-(2-Chloro-4-trifluoromethyl-phenyl)-5-methyl-1H-[1,2,3]triazol-4-yl]-phenol (Compound 3)

To a stirred solution of Compound 2 (0.400 g, 1.0877 mmol) in anhydrous dichloromethane (30 ml), cooled to −78° C. and under a nitrogen flow, a solution of boron tribromide (1.900 g, ≠0.72 ml, 7.6139 mmol) in 5 ml of anhydrous dichloromethane is added drop-wise. The mixture is allowed to reach room temperature spontaneously overnight and it is then cooled again in an ice-salt bath and the excess of the reagent is decomposed upon drop-wise addition of 12 ml of methanol and 12 ml of water. After 5 min stirring, 10% sodium hydroxide solution (15 ml) is added and the aqueous layer, once separated, is acidified with 10% hydrochloric acid solution and extracted with chloroform (3×150 ml). The combined organic layers are dried over MgSO₄, filtered and evaporated to afford a solid residue (0.380 g), which is triturated with petroleum ether, decanted and dried, to afford the title compound as an off-white solid (0.350 g, 91% yield). M.p. 175.6-176.6° C. LC-ESI-HRMS of [M+H]+ shows 354.0634 Da. Calc. 354.062099 Da, dev. 3.7 ppm.

5-Bromomethyl-1-(2-chloro-4-trifluoromethyl-phenyl)-4-(4-methoxy-phenyl)-1H-[1,2,3]triazole (Compound 4)

To a stirred solution of Compound 2 (2.500 g, 6.798 mmol) in carbon tetrachloride (80 ml), N-bromosuccinimide (1.940 g, 10.197 mmol) and a catalytic amount of benzoyl peroxide (0.250 g, 2.0639) are added and the resulting reaction mixture is refluxed overnight. The reaction mixture is cooled to room temperature and solid formed is filtered, and the filtrate is evaporated, to afford a yellow solid (˜3 g, 97% mass balance). The crude residue is purified by column chromatography over silica gel (230-400 mesh), eluting with 9% ethyl acetate in petroleum ether, to obtain the title compound as a yellow solid (2.5 g, 76% yield). M.p. 152.4-153.9° C. LC-ESI-HRMS of [M+H]+ shows 445.9881 Da. Calc. 445.988262 Da, dev. −0.4 ppm.

C-[3-(2-Chloro-4-trifluoromethyl-phenyl)-5-(4-methoxy-phenyl)-3H-[1,2,3]triazol-4-yl]-methylamine (Compound 5)

To a solution of Compound 4 (0.900 g, 2.015 mmol) in absolute ethanol (20 ml) at −20° C., ammonia gas was purged through for 25 min and the reaction mixture is allowed to attain room temperature spontaneously. The resulting reaction mixture is evaporated to dryness, to afford a yellow solid (0.77 g, 100% mass balance). The crude residue is purified by column chromatography over silica gel (230-400 mesh), eluting with 40% ethyl acetate in petroleum ether, to obtain the title compound as an off white solid (0.650 g, 84% yield). LC-ESI-HRMS of [M+H]+ shows 383.0898 Da. Calc. 383.088648 Da, dev. 3 ppm.

4-[5-Aminomethyl-1-(2-chloro-4-trifluoromethyl-phenyl)-1H-[1,2,3]triazol-4-yl]-phenol (Compound 6)

To a stirred solution of Compound 5 (0.150 g, 0.3762 mmol) in anhydrous dichloromethane (25 ml), cooled to 0° C. and under a nitrogen flow, a solution of boron tribromide (0.25 ml, 2.6334 mmol) in 5 ml of anhydrous dichloromethane is added drop-wise. Stirring is continued for 1 hour at 0° C. and one hour at room temperature. The resulting mixture is cooled again in an ice-salt bath and the excess of the reagent is decomposed upon treatment with 3 ml of methanol and 3 ml of water, which are added drop-wise to the reaction mixture. After 5 min stirring, 10% sodium hydroxide solution (8 ml) is added and the aqueous layer, once separated, is acidified with 10% hydrochloric acid solution and extracted with chloroform (3×50 ml). The combined organic layers are dried over MgSO₄, filtered and evaporated to afford a greenish solid (0.130 g). This crude residue is purified by column chromatography over silica gel (230-400 mesh), eluting with 40% ethyl acetate in petroleum ether, to obtain the title compound as a yellow solid (0.096 g, 69% yield). M.p. 96.5-98.9° C. LC-ESI-HRMS of [M+H]+ shows 369.0744 Da. Calc. 369.072998 Da, dev. 3.8 ppm.

[3-(2-Chloro-4-trifluoromethyl-phenyl)-5-(4-methoxy-phenyl)-3H-[1,2,3]triazol-4-yl]-methanol (Compound 7)

To a solution of Compound 4 (0.900 g, 2.015 mmol) in water (80 ml) and dioxane (80 ml), calcium carbonate (0.3025 g, 3.0225 mmol) is added and the mixture is refluxed overnight. The resulting reaction mixture is cooled to room temperature and is extracted with ethyl acetate (3×150 ml). The combined organic layers are dried over MgSO₄, filtered and evaporated to afford a yellow gummy material (0.773 g, 100% mass balance). The crude residue is purified by column chromatography over silica gel (230-400 mesh), eluting with 30% ethyl acetate in petroleum ether, to obtain the title compound as a light yellow solid (0.600 g, 69% yield). M.p. 130.8-132.2° C. LC-ESI-HRMS of [M+H]+ shows 384.0732 Da. Calc. 384.072664 Da, dev. 1.4 ppm.

4-[1-(2-Chloro-4-trifluoromethyl-phenyl)-5-hydroxymethyl-1H-[1,2,3]triazol-4-yl]-phenol (Compound 8)

To a stirred solution of Compound 7 (0.300 g, 0.7817 mmol) in anhydrous dichloromethane (25 ml), cooled to −20° C. and under a nitrogen flow, a 5 solution of boron tribromide (1.371 g, ˜0.52 ml, 5.4719 mmol) in 5 ml of anhydrous dichloromethane is added drop-wise. Stirring is continued for 1 hour at −20° C. and at room temperature overnight. The resulting mixture is cooled again in an ice-salt bath and the excess of the reagent is decomposed upon treatment with 8 ml of methanol and 8 ml of water, which are added drop-wise to the reaction mixture. After 5 min stirring, 10% sodium hydroxide solution (10 ml) is added and the aqueous layer, once separated, is acidified with 10% hydrochloric acid solution and extracted with chloroform (3×100 ml). The combined organic layers are dried over MgSO₄, filtered and evaporated to afford a solid residue (0.280 g), which is triturated with petroleum ether, decanted and dried, to afford the title compound as an off-white solid (0.243 g, 83% yield). M.p. 180.6-182.2° C. LC-ESI-HRMS of [M+H]+ shows 370.0569 Da. Calc. 370.057014 Da, dev. −0.3 ppm.

1-(2-Fluoro-4-trifluoromethyl-phenyl)-4-(4-methoxy-phenyl)-1H-[1,2,3]triazole (Compound 9)

A solution of freshly-prepared 1-azido-2-fluoro-4-trifluoromethyl-benzene (3.000 g, 14.626 mmol) and commercial 1-ethynyl-4-methoxybenzene (2.320 g, 17.551 mmol) in ethanol (60 ml) is refluxed at 80° C. for 24 hr, followed by evaporation to dryness and addition of water (150 ml). This resulting mixture is extracted with ethyl acetate (3×300 ml), and the combined organic layers are dried over MgSO₄, filtered and evaporated, to afford a dark brown oily residue (˜4.9 g, 99% mass balance). The crude residue containing a mixture of the two regioisomers (1,4 and 1,5 diarylsubstituted triazoles) is purified by column chromatography over silica gel (60-120 mesh), eluting with 2-4% ethyl acetate in hexane, to obtain the title compound as an off-white solid (0.900 g, 18% yield). M.p. 152.3-153.5° C. LC-ESI-HRMS of [M+H]+ shows 338.0912 Da. Calc. 338.091104 Da, dev. 0.3 ppm.

4-[1-(2-Fluoro-4-trifluoromethyl-phenyl)-1H-[1,2,3]triazol-4-yl]-phenol (Compound 10)

To a stirred solution of Compound 8 (0.570 g, 1.690 mmol) in anhydrous dichloromethane (15 ml), cooled to −78° C. and under a nitrogen flow, a solution of boron tribromide (2.964 g, ˜1.1 ml, 11.83 mmol) in 5 ml of anhydrous dichloromethane is added drop-wise. The mixture is allowed to reach room temperature spontaneously and stirring is then continued at room temperature (5 hours in total). The resulting mixture is cooled again in an ice-salt bath and the excess of the reagent is decomposed upon drop-wise addition of 20 ml of methanol and 20 ml of water. After 5 min stirring, 5% sodium bicarbonate solution (25 ml) is added and the resulting mixture extracted with chloroform (3×200 ml). The combined organic layers are dried over MgSO₄, filtered and evaporated to afford the title compound as an off-white solid (0.540 g, 99% yield). M.p. 214.8-215.7° C. LC-ESI-HRMS of [M+H]+ shows 324.074715043693 Da. Calc. 324.075454 Da, dev. −2.3 ppm.

5-Chloro-4-(2-chloro-4-methoxy-phenyl)-1-(2-fluoro-4-trifluoromethyl-phenyl)-1H-[1,2,3]triazole (Compound 11)

To an ice-cold suspension of 5-(2-chloro-4-methoxy-phenyl)-3-(2-fluoro-4-trifluoromethyl-phenyl)-3H-[1,2,3]triazol-4-ylamine (0.650 g, 1.6807 mmol) (prepared by following the general procedure described in WO 2009/019278) in ethanol (99%) (20 ml), dry HCl gas is gently bubbled through. Isoamyl nitrite (0.394 g, 3.362 mmol) is added and the reaction mixture is stirred at 0-5° C. for 12 hours and evaporated to dryness. Water (100 ml) is added and the new mixture is extracted with dichloromethane (3×150 ml). The combined organic layers are dried over MgSO₄, filtered and evaporated to afford a crude residue. This residue is purified by column chromatography over silica gel (60-120 mesh), eluting with 4% ethyl acetate in hexane, to obtain the title compound as a white solid (0.260 g, 38% yield). M.p. 95.8-97.5° C.

3-Chloro-4-[5-chloro-1-(2-fluoro-4-trifluoromethyl-phenyl)-1H-[1,2,3]triazol-4-yl]-phenol (Compound 12)

To a stirred solution of Compound 10 (0.150 g, 0.3693 mmol) in anhydrous dichloromethane (5 ml), cooled to −78° C. and under a nitrogen flow, a solution of boron tribromide (0.648 g, ˜0.25 ml, 2.5851 mmol) in 5 ml of anhydrous dichloromethane is added drop-wise. The mixture is allowed to reach room temperature spontaneously and stirring is then continued at room temperature (8 hours in total). The mixture is cooled again in an ice-salt bath and the excess of the reagent is decomposed upon drop-wise addition of 5 ml of methanol and 5 ml of water. After 5 min stirring, 5% sodium bicarbonate solution (10 ml) is added and the resulting mixture extracted with dichloromethane (3×25 ml). The combined organic layers are dried over MgSO₄, filtered and evaporated to afford the title compound as a grey solid (0.101 g, 69% yield). M.p. 150.2-151.5° C.

### Example 2

### Biological Activity

In this example the positive modulation of wild-type nAChR α7 receptors by Compounds 3, 7 and 8, representative of the invention, was determined using nAChR a7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent test applications, increasing concentrations (0.01-31.6 μM) of the test compound were applied 30 s before and during the acetylcholine (100 μM) application, which caused a robust increase in the acetylcholine-induced current amplitude.

The positive modulation in the presence of Compound 4 was calculated as (test-control)/control×100% and the concentration response curve for this positive modulation was fitted to the sigmoidal logistic equation: I=Iₘₐₓ/(1+(EC₅₀/[compound])ⁿ), where Iₘₐₓ represents the maximal modulation of the control response, EC₅₀ is the concentration causing a half maximal response, and n is the slope coefficient.

The calculated EC₅₀ values for Compounds 3, 7 and 8 were 14, 17 and 11 μM, respectively, and the calculated EC₅₀ Iₘₐₓ values for Compounds 3, 7 and 8 were 119, 128 and 136%, respectively. This is an indication of a biological activity as potent modulators of the nicotinic acetylcholine α7 receptor subtype.

1. A diphenyl 1,2,3-triazole derivative represented by Formula I 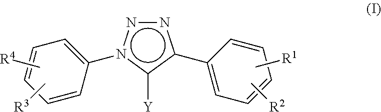
a stereoisomer thereof or a mixture of its stereoisomers, or a pharmaceutically acceptable salt thereof, wherein
Y represents hydrogen, halo, alkyl, halo-alkyl, hydroxy-alkyl or amino-alkyl; and
R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

2. The diphenyl 1,2,3-triazole derivative of claim 1, a stereoisomer thereof or a mixture of its stereoisomers, or a pharmaceutically acceptable salt thereof, wherein
Y represents alkyl, halo-alkyl, hydroxy-alkyl or amino-alkyl.

3. The diphenyl 1,2,3-triazole derivative of either one of claims claim 1, a stereoisomer thereof or a mixture of its stereoisomers, or a pharmaceutically acceptable salt thereof, wherein
R¹, R², R³ and R⁴, independently of each other, represent a substituent selected from the group consisting of hydrogen, alkyl, halo, trifluoromethyl, trifluoromethoxy, cyano, alkoxy, hydroxy, amino, N-(alkyl-carbonyl)-amino, sulfamoyl and oxadiazolyl.

4. The diphenyl 1,2,3-triazole derivative of claim 1, which is
1-(2-Fluoro-4-trifluoromethyl-phenyl)-4-(4-methoxy-phenyl)-5-methyl-1H-[1,2,3]triazole;
1-(2-Chloro-4-trifluoromethyl-phenyl)-4-(4-methoxy-phenyl)-5-methyl-1H-[1,2,3]triazole;
4-[1-(2-Chloro-4-trifluoromethyl-phenyl)-5-methyl-1H-[1,2,3]triazol-4-yl]-phenol;
5-Bromomethyl-1-(2-chloro-4-trifluoromethyl-phenyl)-4-(4-methoxy-phenyl)-1H-[1,2,3]triazole;
C-[3-(2-Chloro-4-trifluoromethyl-phenyl)-5-(4-methoxy-phenyl)-3H-[1,2,3]triazol-4-yl]-methylamine;
4-[5-Aminomethyl-1-(2-chloro-4-trifluoromethyl-phenyl)-1H-[1,2,3]triazol-4-yl]-phenol;
[3-(2-Chloro-4-trifluoromethyl-phenyl)-5-(4-methoxy-phenyl)-3H-[1,2,3]triazol-4-yl]-methanol;
4-[1-(2-Chloro-4-trifluoromethyl-phenyl)-5-hydroxymethyl-1H-[1,2,3]triazol-4-yl]-phenol;
1-(2-Fluoro-4-trifluoromethyl-phenyl)-4-(4-methoxy-phenyl)-1H-[1,2,3]triazole;
4-[1-(2-Fluoro-4-trifluoromethyl-phenyl)-1H-[1,2,3]triazol-4-yl]-phenol;
5-Chloro-4-(2-chloro-4-methoxy-phenyl)-1-(2-fluoro-4-trifluoromethyl-phenyl)-1H-[1,2,3]triazole; or
3-Chloro-4-[5-chloro-1-(2-fluoro-4-trifluoromethyl-phenyl)-1H-[1,2,3]triazol-4-yl]-phenol;
a stereoisomer thereof or a mixture of its stereoisomers, or a pharmaceutically acceptable salt thereof.

5. A pharmaceutical composition comprising a therapeutically effective amount of a diphenyl 1,2,3-triazole derivative of claim 1, or a stereoisomer thereof or a mixture of its stereoisomers, or a pharmaceutically acceptable addition salt thereof, together with at least one pharmaceutically acceptable carrier or diluent.

6. (canceled)

7. The method according to claim 8, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.

8. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a diphenyl 1,2,3-triazole derivative of claim 1 or a stereoisomer thereof or a mixture of its stereoisomers, or a pharmaceutically acceptable salt thereof.
